(19) United States
(12) Patent Application Publication
(10) Pub. No.: US 2024/0341983 A1
(43) Pub. Date: Oct. 17, 2024

(54) SYSTEMS AND METHODS FOR TREATMENT OF INTRACRANIAL STENOSIS

(71) Applicant: Covidien LP, Mansfield, MA (US)

(72) Inventors: Varun Umesh Kashyap, Palo Alto, CA (US); Mark P. Ashby, Laguna Niguel, CA (US); Bala Subramanya Pavan Kumar Kandala, Irvine, CA (US); Kamel M. Chair, Hayward, CA (US); Alex G. Garcia, Aliso Viejo, CA (US)

(21) Appl. No.: 18/633,039

(22) Filed: Apr. 11, 2024

Related U.S. Application Data
(60) Provisional application No. 63/496,626, filed on Apr. 17, 2023.

(51) Int. Cl. A61F 2/95; A61B 5/00; A61B 5/0215

(52) U.S. Cl. CPC A61F 2/95; A61B 5/02158; A61B 5/6852; A61B 2505/05; A61F 2002/9505

(57) ABSTRACT

A system for treatment of intracranial stenosis can include a catheter having a lumen, and an elongate member extending through the lumen. A distal pressure sensor is coupled to a distal end portion of the elongate member and is configured to obtain blood pressure parameters to characterize blood flow at a treatment site before and after treatment. A stent is disposed within the catheter lumen in a radially constrained configuration. The stent surrounds the elongate member such that the distal end portion of the elongate member is disposed distal to a distal end of the stent. The stent is configured to radially expand into apposition with the blood vessel to increase blood flow at the treatment site when released from the catheter.

### CROSS-REFERENCE TO RELATED APPLICATIONS

This application claims the benefit of and priority to U.S. Provisional Patent Application Ser. No. 63/496,626 filed Apr. 17, 2023, the entire disclosure of which is incorporated by reference herein.

### TECHNICAL FIELD

The present technology relates generally to systems and methods for restoring patency to bodily lumens. Some embodiments of the present technology relate to systems and methods for treatment of intracranial stenosis.

### BACKGROUND

Idiopathic intracranial hypertension (IIH) or pseudotumor cerebri—a condition that affects around 100,000 Americans each year—is characterized by persistently increased intracranial pressure. Not all instances of persistent increased intracranial pressure are IIH, however. For example, there may be a mass causing the increased intracranial pressure or CSF not draining or being absorbed properly (i.e. hydrocephalus). Symptoms of IIH include headaches, pulse-synchronous tinnitus, diplopia, visual field loss/amputations of varying degrees resulting from papilledema, and other visual disturbances including blurred vision and transient visual obscurations. Other non-specific symptoms such as numbness of the extremities, generalized weakness, anosmia and incoordination may be present in these patients.

Imaging studies of these conditions have found normal or small ventricles and normal brain parenchyma based on computed tomography (CT) or magnetic resonance imaging (MRI) data. MRI typically shows flattening of the posterior aspect of the globes, dilatation of the optic nerve sheathes with elevation of the optic disc sometimes visible on axial T2-weighted MRI images, and occasionally an acquired Chiari malformation. To qualify as IIH, there must be no mass lesion or any obvious cause for elevated intracranial pressure. In cases of IIH, other features of chronically raised intracranial pressure such as a partially empty sella are frequently present, and cerebrospinal fluid leaks into the sella turcica are sometimes seen.

Magnetic resonance venography (MRV) studies indicate that venous sinus stenoses are frequently present in patients with IIH. The goal of treatment in such cases is to preserve vision and manage headache. Traditional medical management involves administration of medication, such as acetazolamide, which reduces the rate of production of cerebrospinal fluid, topiramide, and occasionally furosemide. Patients are also typically advised to lose weight, with the aim of decreasing central venous pressure. Lumbar puncture with 20-30 ml cerebrospinal fluid drainage typically produces immediate relief of symptoms, which may last significantly longer than the amount of time required for the body to replace the volume of cerebrospinal fluid withdrawn. Surgical therapy includes diversion techniques such as optic nerve sheath fenestration, lumboperitoneal shunting or ventriculoperitoneal or ventriculoatrial shunting aimed at a fast, dependable reduction in intracranial pressure. Although these treatments work well initially, in the long run there is a risk of failure or requirement of re-treatment. Shunt procedures are frequently associated with technical failures (blockage, kinking, disconnection, or dislodgement, infection) resulting in a need for shunt revision in ≥30% of patients per year.

Recently, it has been found that many patients with IIH harbor stenosis along the transverse-sigmoid sinus junction, either bilaterally or in the dominant sinus. These venous stenoses may be due to an intrinsic abnormality in the sinus wall, such as in an arachnoid granulation, scar tissue or septation, in which case they appear as a focal region of stenosis, or may be extrinsic, occurring as a result of compression by elevated intracranial pressure, in which case there is a more tapered appearance. In some studies, IIH patients have been treated by implanting stents originally designed for carotid or peripheral vascular use. Such implants have been successful in achieving symptomatic improvement, with improvement or resolution of headaches, pulse-synchronous tinnitus, diplopia, and transient visual obscuration. While use of such stents has shown promising results, there remains a need for treatment systems designed specifically for the intracranial venous context, including systems and methods for verifying, treating, and confirming venous sinus stenosis in patients suffering from IIH and other such conditions.

### SUMMARY

The present technology is illustrated, for example, according to various aspects described below. These are provided as examples and do not limit the present technology.

Clause 1. A method for treatment of intracranial stenosis comprising: disposing a catheter within a blood vessel such that a distal end of the catheter is disposed proximal to a treatment site, the catheter defining a lumen containing a stent in a radially compressed configuration; disposing a pressure sensor at or distal to a distal end of the catheter at a proximal measurement site that is proximal to the treatment site; obtaining a pre-treatment proximal blood pressure parameter via the pressure sensor at the proximal measurement site; disposing a pressure sensor at a distal measurement site that is distal to the treatment site; obtaining a pre-treatment distal blood pressure parameter via the pressure sensor at the distal measurement site; deploying the stent by: while the distal end of the catheter is disposed at or distal to the treatment site, proximally retracting the catheter relative to the stent, thereby releasing the stent from the catheter; and allowing the stent to self-expand into apposition with the blood vessel at the treatment site; obtaining a post-treatment distal blood pressure parameter via a pressure sensor at the distal measurement site; and obtaining a post-treatment proximal blood pressure parameter via a pressure sensor at the proximal measurement site.

Clause 2. The method of clause 1, wherein a single pressure sensor is used to obtain the pre-treatment proximal blood pressure parameter, the post-treatment proximal blood pressure parameter, the pre-treatment distal blood pressure parameter, and the post-treatment distal blood pressure parameter, wherein the pressure sensor is coupled to an elongate member slidably extending through the catheter lumen.

Clause 3. The method of any one of the preceding clauses, wherein obtaining the pre-treatment distal blood pressure parameter comprises distally advancing the elongate member such that the pressure sensor is moved from the proximal measurement site to the distal measurement site.

Clause 4. The method of any one of the preceding clauses, wherein obtaining the post-treatment proximal blood pressure parameter comprises proximally retracting the elongate member such that the pressure sensor is moved from the distal measurement site to the proximal measurement site.

Clause 5. The method of any one of the preceding clauses, wherein: a proximal pressure sensor is used to obtain the pre-treatment proximal blood pressure parameter and to obtain the post-treatment proximal blood pressure parameter, a separate distal pressure sensor is used to obtain the pre-treatment distal blood pressure parameter and the post-treatment distal blood pressure parameter, and the distal pressure sensor is coupled to an elongate member slidably extending through the catheter lumen.

Clause 6. The method of any one of the preceding clauses, wherein the proximal pressure sensor is carried by the catheter.

Clause 7. The method of any one of the preceding clauses, wherein the proximal pressure sensor is carried by the elongate member at a position proximal to the distal pressure sensor.

Clause 8. The method of any one of the preceding clauses, further comprising: comparing the pre-treatment and post-treatment blood pressure parameters; and based on the comparison, re-positioning the stent within the blood vessel.

Clause 9. A system for treatment of intracranial stenosis comprising: a catheter defining a lumen and having a distal end portion configured to be disposed adjacent to a treatment site within an intracranial blood vessel; an elongate member extending through the catheter lumen; a distal pressure sensor coupled to a distal end portion of the elongate member, the distal pressure sensor configured to obtain blood pressure parameters to characterize blood flow at the treatment site before and after treatment; and a stent disposed within the catheter lumen in a radially constrained configuration, the stent surrounding the elongate member such that the distal end portion of the elongate member is disposed distal to a distal end of the stent, wherein the stent is configured to radially expand into apposition with the blood vessel to increase blood flow at the treatment site when released from the catheter.

Clause 10. The system of clause 9, further comprising a proximal pressure sensor configured to obtain blood pressure parameters in conjunction with the distal pressure sensor to characterize blood flow at the treatment site before and after treatment.

Clause 11. The system of clause 10, wherein the proximal pressure sensor is carried by the elongate member at a position proximal to the distal pressure sensor.

Clause 12. The system of clause 10, wherein the proximal pressure sensor is carried by the catheter.

Clause 13. The system of any one of clauses 9-12, further comprising: a second elongate member extending through the catheter lumen; and a proximal pressure sensor coupled to a distal end portion of the second elongate member, the proximal pressure sensor configured to obtain blood pressure parameters in conjunction with the distal pressure sensor to characterize blood flow at the treatment site before and after treatment.

Clause 14. The system of any one of clauses 9-13, further comprising: a pusher member releasably coupled to the stent and extending through the catheter lumen; and a second sheath disposed within the catheter lumen, the second sheath surrounding the pusher member and at least a proximal portion of the elongate member.

Clause 15. A method of treating intracranial stenosis with the system of claim 9, the method comprising: distally advancing the catheter carrying the elongate member and the stent through a patient's vasculature to a position proximal to treatment site within an intracranial blood vessel, such that the distal pressure sensor extends beyond a distal end of the catheter and is disposed at a proximal measurement site that is proximal to the treatment site; obtaining a pre-treatment proximal blood pressure parameter via the distal pressure sensor at the proximal measurement site; distally advancing the catheter and the elongate member to position the distal pressure sensor at a distal measurement site distal that is distal to the treatment site; obtaining a pre-treatment distal blood pressure parameter via the distal pressure sensor at the distal measurement site; deploying the stent by proximally retracting the catheter relative to the stent, thereby releasing the stent from the catheter such that the stent self-expands into apposition with the blood vessel at the treatment site; after deploying the stent, obtaining a post-treatment distal blood pressure parameter via the distal pressure sensor at the distal measurement site; proximally retracting the elongate member to position the distal pressure sensor at the proximal measurement site; and obtaining a post-treatment proximal blood pressure parameter via the distal pressure sensor at the proximal measurement site.

Clause 16. The method of clause 15, wherein the stent is releasably coupled to a pusher member, the method further comprising, after obtaining the post-treatment proximal blood pressure parameter and obtaining the post-treatment distal blood pressure parameter, releasing the stent from the pusher member.

Clause 17. A method of treating intracranial stenosis with the system of claim 9, the method comprising: distally advancing the catheter carrying the elongate member and the stent through a patient's vasculature to a treatment site within an intracranial blood vessel, such that the distal pressure sensor extends beyond a distal end of the catheter and is disposed at a proximal measurement site that is proximal to the treatment site; obtaining a pre-treatment proximal blood pressure parameter via the distal pressure sensor at the proximal measurement site; distally advancing the catheter and the elongate member to position the distal pressure sensor at a distal measurement site distal that is distal to the treatment site; obtaining a pre-treatment distal blood pressure parameter via the distal pressure sensor at the distal measurement site; deploying the stent by proximally retracting the catheter relative to the stent, thereby releasing the stent from the catheter such that the stent self-expands into apposition with the blood vessel at the treatment site; after deploying the stent, obtaining a post-treatment distal blood pressure parameter via the distal pressure sensor at the distal measurement site; disposing a proximal pressure sensor at the proximal measurement site; and after deploying the stent, obtaining a post-treatment proximal blood pressure parameter via the proximal pressure sensor at the proximal measurement site.

Clause 18. The method of clause 17, wherein the proximal pressure sensor is carried by the elongate member at a position proximal to the distal pressure sensor.

Clause 19. The method of clause 17, wherein the proximal pressure sensor is carried by a second elongate member slidably disposed within the catheter.

Clause 20. The method of clause 17, wherein the proximal pressure sensor is carried by the catheter.

Clause 21. The method of any one of clauses 17-20, wherein the stent is releasably coupled to a pusher member, the method further comprising, after obtaining the post-treatment proximal blood pressure parameter and obtaining the post-treatment distal blood pressure parameter, releasing the stent from the pusher member.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1A shows a perspective view of a treatment system for restoring patency to a bodily lumen in accordance with one or more embodiments of the present technology.

FIG. 1B is a side schematic view of a portion of the treatment system shown in FIG. 1A.

FIG. 2 is a side schematic view of a portion of another example treatment system in accordance with one or more embodiments of the present technology.

FIG. 3 is a side schematic view of a portion of another example treatment system in accordance with one or more embodiments of the present technology.

FIG. 4 is a flow chart illustrating a method of treating intracranial stenosis in accordance with one or more embodiments of the present technology.

FIGS. 5A-5D illustrate an example method of treating intracranial stenosis in using a sensor assembly with multiple sensors in accordance with one or more embodiments of the present technology.

FIGS. 6A-6D illustrate another example method of treating intracranial stenosis in using a sensor assembly with multiple sensors in accordance with one or more embodiments of the present technology.

FIGS. 7A-7E illustrate another example method of treating intracranial stenosis in using a sensor assembly with a single sensor in accordance with one or more embodiments of the present technology.

### DETAILED DESCRIPTION

Disclosed herein are examples of systems and methods for restoring patency to bodily lumens such as blood vessels. As noted above, idiopathic intracranial hypertension (IIH) or pseudotumor cerebri is a condition that affects many thousands of patients worldwide. Recently, it has been found that many IIH patients exhibit stenosis along the transverse-sigmoid sinus junction. Although off-label carotid, peripheral, and other stents have been used to treat venous stenosis, it can be difficult for clinicians to localize the appropriate treatment site within the vessel and to confirm efficacy of the intervention. For instance, a clinician may need to advance a separate intravascular pressure sensor system to the treatment site to characterize blood pressure and/or flow adjacent to the stenosis. Next, the clinician removes the pressure sensor system and separately advances a stent to the treatment site for deployment at the stenosis. Finally, the stent delivery system is removed, and the pressure sensor system is re-introduced to evaluate blood flow at the treatment site to confirm that the treatment has been effective. If it has not been effective, the stent may need to be repositioned or another stent may need to be deployed at another location.

Given the relatively high patient population suffering from IIH, treatment systems designed specifically for this application provide several benefits, including implant sizing recommendations, improved case of use, confirmation of efficacy, and real-time assessment of the need for intraprocedural repositioning and/or implant replacement. The present technology provides a treatment system with a pressure sensor assembly including one or more pressure sensors to evaluate blood pressure and/or flow at the treatment site and an implant delivery system configured to deliver an implant to the treatment site for deployment at the stenosis. In operation, the pressure sensor(s) and the implant can be advanced together to the treatment site, where the pressure sensor(s) can obtain one or more pre-treatment measurements to characterize blood pressure and/or flow at the treatment site. These measurements can confirm the presence of stenosis and impeded blood flow. The implant may then be at least partially deployed, and one or more post-treatment measurements of blood pressure and/or flow can be made via the pressure sensor(s). If these measurements confirm that blood flow has been improved and/or restored, the implant can be fully released and the treatment system can be removed from the body. If these post-treatment measurements indicate that blood flow has not been sufficiently improved and/or restored, then the implant may be resheathed and repositioned, the implant may be removed and replaced with a more suitable implant, or an additional implant may be introduced in addition to the first implant. In any of these cases, further post-treatment measurements can be obtained and used to confirm efficacy of the intervention before removal of the treatment system from the patient.

In various examples, the measurements obtained via the sensor(s) (e.g., characterizing blood flow, blood pressure, etc.) can be collected and analyzed via one or more computing devices. In some instances, the data can be collected and aggregated across many patients (using appropriate anonymization techniques as needed) to evaluate pre-, intra-, and post-procedural patient conditions. Such data may be utilized as input to a machine learning algorithm for predicting or recommending appropriate implant size or other characteristics, patient outcomes, identifying potential complications, or other parameters that may impact procedural decisions and patient outcomes.

The present technology provides devices, systems, and methods for restoring patency to a blood vessel lumen, such as at the site of a venous stenosis. Although many of the embodiments are described below with respect to devices, systems, and methods for treating a cerebral or intracranial stenosis, other applications and other embodiments in addition to those described herein are within the scope of the technology. For example, the treatment systems and methods of the present technology may be used to restore patency to bodily lumens other than blood vessels (e.g., the digestive tract, etc.) and/or may be used to restore patency to blood vessels outside of the brain (e.g., pulmonary, abdominal, cervical, or thoracic blood vessels, or peripheral blood vessels including those within the legs or arms, etc.).

### I. Select Embodiments of Treatment Systems

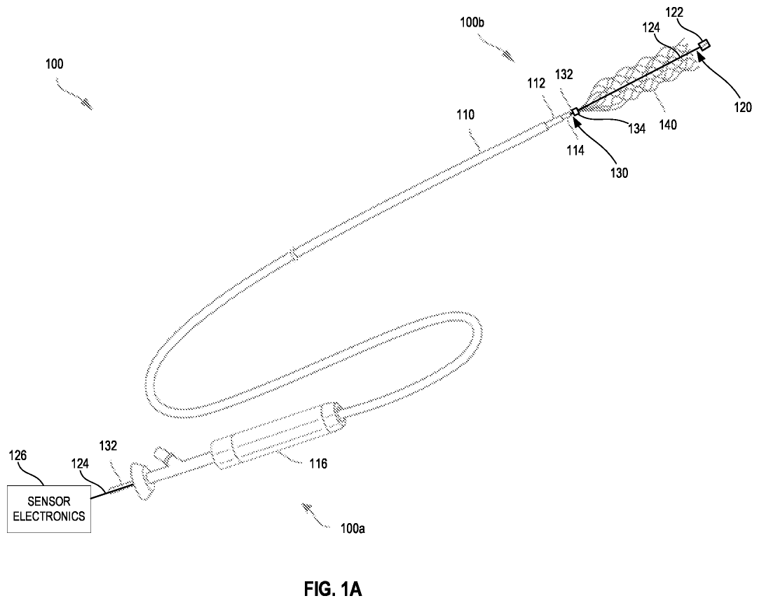

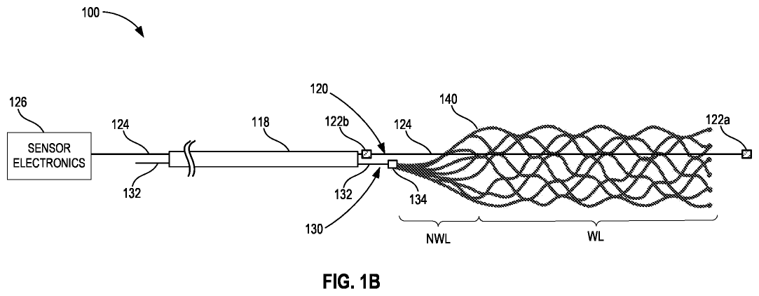

FIG. 1A illustrates a perspective view of a treatment system 100 according to one or more embodiments of the present technology, and FIG. 1B illustrates a schematic side view of a portion of the treatment system 100. As shown in FIG. 1A, the treatment system 100 can include a plurality of catheters 110, 112, and 114, a sensor assembly 120, and an implant delivery assembly 130 configured to deliver an implant 140 (e.g., a stent) to a treatment site (e.g., an intravascular treatment site). In operation, the catheters 110, 112, and 114 can be arranged coaxially and be configured to slidably receive components of the sensor assembly 120 and/or the implant delivery assembly 130 therethrough. The treatment system 100 has a proximal portion 100a configured to be positioned outside the patient's body and a distal portion 100b configured to be intravascularly positioned within a blood vessel (such as an intracranial blood vessel) at a treatment site (e.g., at or proximate a stenosis).

In some embodiments, such as that shown in FIG. 1A, the treatment system 100 includes a first catheter 110 (such as a balloon guide catheter), a second catheter 112 (such as a distal access catheter or aspiration catheter) configured to be slidably disposed within a lumen of the first catheter 110, and a third catheter 114 (such as a microcatheter or delivery sheath) configured to be slidably disposed within a lumen of the second catheter 112. At least a portion of the sensor assembly 120 and the implant delivery assembly 130 can slidably extend through the lumen of the third catheter 114. In the illustrated configuration, the first catheter 110 can be coupled at its proximal end portion to a handle 116.

According to some embodiments, the bodies of the catheters 110, 112, and 114 can be made from various thermoplastics, e.g., polytetrafluoroethylene (PTFE or TEFLON®), fluorinated ethylene propylene (FEP), high-density polyethylene (HDPE), polyether ether ketone (PEEK), etc., which can optionally be lined on the inner surface of the catheters or an adjacent surface with a hydrophilic material such as polyvinylpyrrolidone (PVP) or some other plastic coating. Additionally, either surface can be coated with various combinations of different materials, depending upon the desired results.

The sensor assembly 120 includes one or more sensors 122 which are electrically coupled, via an elongate lead 124, to sensor electronics 126. In the illustrated configuration of FIG. 1A, the sensor(s) 122 are disposed distal to the catheters 110, 112, 114, and the lead 124 extends through the lumen of the third catheter 114 between the sensor(s) 122 and the sensor electronics 126. In the example shown in FIG. 1B, the sensor assembly 120 includes a distal sensor 122a and a proximal sensor 122b, each of which are coupled to the lead 124. In the configuration shown in FIG. 1B, the distal sensor 122a is disposed distal to the implant 140, and the proximal sensor 122b is disposed proximal to the implant 140. In operation, the lead 124 can be slidable relative to the catheter 114, such that the particular position and/or orientation of each of the sensors 122a and 122b can vary as the clinician slidably advances or retracts the lead 124 relative to the other components of the treatment system 100.

The sensor(s) 122 are configured to be disposed at or adjacent a treatment site (e.g., at or adjacent an intracranial stenosis), and may be used to obtain measurements of blood pressure, blood flow, other parameters characterizing fluid movement adjacent the sensor(s) 122, or any other suitable physiological parameters. In various examples, the number, placement, type, and configuration of sensors 122 can vary. For instance, a single sensor 122 may be coupled to the lead 124, or multiple sensors 122 can be coupled to the lead 124, in which case the lead 124 may include multiple discrete conductive paths to allow signals from the various sensors to be communicated separately to the sensor electronics 126. In some implementations, multiple discrete leads 124, each having one or more sensors 122 coupled thereto, can be provided. In operation, the sensors 122 can be disposed at various locations relative to the treatment site (e.g., a venous stenosis) and/or relative to the implant 140. Measurements taken at different times and/or at different locations can be analyzed and evaluated (e.g., via the sensor electronics 126) to characterize blood pressure, blood flow, and/or other physiological parameters at or adjacent to the treatment site.

In various examples, the sensor(s) 122 may be MEMS-based, resistive, capacitive, piezoelectric, optical, ultrasonic transducers or any combination thereof. For example, an ultrasound sensor may utilize Doppler ultrasound techniques to measure blood flow velocity. Such a sensor can generate high-frequency sound waves that are transmitted into the blood vessel. The reflected waves are then detected by the sensor, and the frequency shift is used to calculate blood flow velocity. This information may then be used to derive or estimate blood flow and/or pressure. In some instances, the sensor(s) 122 can be MEMS-based sensors, for example including a MEMS pressure transducer that converts blood pressure into an electrical signal. Additionally or alternatively, the sensor(s) 122 can be optical sensors configured to use optical techniques, such as laser Doppler flowmetry or diffuse correlation spectroscopy, to measure blood flow. Such a sensor can emit light into the blood vessel, and the scattered light is then detected and analyzed to determine blood flow velocity and/or volume.

The sensor electronics 126 can be positioned outside the body and placed in electrical communication with the sensor(s) 122 via the lead 124. Additionally or alternatively, the sensor(s) 112 can be configured to wirelessly communicate with the sensor electronics 126, rather than or in addition to sending electrical signals along the lead 124. In some examples, the lead 124 takes the form of an electrically conductive wire, shaft, or other structure configured to carry electrical signals between the sensor(s) 122 and the sensor electronics 126. In some instances, the lead 124 can have a coating, jacket, or other structure that electrically insulates the outer surface of the lead 124 along at least a portion of its length. For example, an electrically insulating layer or material can surround the lead 124 along some or all of its length. The insulating material can be, for example, PTFE (polytetrafluoroethylene or TEFLON™) or any other suitable electrically insulating coating (e.g., polyimide, oxide, ETFE-based coatings, or any suitable dielectric polymer). In some embodiments, the insulating material extends along substantially the entire length of the lead 124. In some embodiments, the insulating material does not cover the proximal-most portion of the lead 124, providing an exposed region of the lead 124 to which the sensor electronics 126 can be electrically coupled. In some embodiments, for example, the insulating material terminates proximally at the proximal terminus of the lead 124, and the electronics 126 can electrically couple to the lead 124 at its proximal terminus, for example using a coaxial connector.

In some implementations, the lead 124 is configured to be slidably advanced through a catheter (e.g., third catheter 114) to the treatment site. For instance, a clinician gripping the proximal end portion of the lead 124 can distally advance or proximally retract the lead 124 relative to a surrounding catheter, thereby manipulating a position of the sensor(s) 112 relative to the surrounding catheter. In at least some variations, the lead 124 can be arranged to extend through a lumen of the implant 140 (e.g., a tubular stent). Alternatively, the lead 124 can be arranged to extend along an outer surface of the implant 140, such that the lead 124 is disposed radially between the implant 140 and the vessel wall when positioned within a blood vessel.

Among examples, the sensor electronics 126 can include components for communicating with the sensor(s) 122 (e.g., transmitting signals to cause the sensor(s) 122 to obtain measurements, receiving measurement signals from the sensor(s) 122, etc.). In some implementations, the sensor electronics 126 may store, analyze, and/or evaluate signals from the sensor(s) 122 to derive, obtain, or generate physiological parameters that characterize fluid flow at the treatment site or other suitable physiological parameters. Example parameters include blood flow, blood volume, and blood pressure, though other physiological parameters may also be used. In various examples, the sensor electronics 126 can include one or more of: a power source (e.g., a battery or connection for facility power), a processor or controller, a data storage component that stores instructions (e.g., in the form of software, code or program instructions executable by the processor), a network interface (e.g., for wired or wireless communication with other computing devices), user interface components (e.g., display, buttons, touch-sensitive surface, microphone, audio transducers, etc.), and/or any other suitable electronic components to facilitate obtaining physiological measurements, analyzing such measurements, and/or transmitting or receiving such information to or from other computing devices.

As described in more detail elsewhere herein, the sensor assembly 120 can be used to obtain pre-and post-treatment measurements of blood pressure and/or blood flow at a treatment site, such as at an intracranial stenosis. These measurements can beneficially enable a clinician to confirm efficacy of a given treatment immediately after deployment of an implant 140 or other intervention.

With continued reference to FIGS. 1A and 1B, the implant delivery assembly 130 includes an elongate core member 132 releasably coupled to the implant 140 via a coupler 134. In various embodiments, the core member 132 can be a solid pushwire, for example a wire made of Nitinol, stainless steel, or other metal or alloy. Additionally or alternatively, the core member 132 can be or include a tubular member, such as a hypotube having flexibility-enhancing features such as slots or spiral cuts formed therein. The core member 132 can have sufficient flexibility, stiffness, column strength, kink-resistance, and/or other material properties to facilitate slidable advancement through tortuous vasculature to an intracranial treatment site, with a clinician grasping a proximal end portion of the core member 132 to slidably advance or retract the core member 132 (and thereby the implant 140) relative to other components of the treatment system 100.

As noted above, the implant 140 is releasably coupled to the core member 132 via coupler 134, which can take the form of an electrolytically severable joint, a mechanically separable joint or connection, or other suitable connection disposed at a distal end portion of the core member 132. The coupler 134 can optionally be radiopaque, for example including platinum or other radiopaque material, thereby enabling visualization of the proximal end of the implant 140 under fluoroscopy. In some embodiments, additional radiopaque markers can be disposed at various locations along the treatment system 100, for example along the catheter(s) 110, 112, and 114, the sensor assembly 120 (e.g., the sensor(s) 122, the lead 124), or the implant 140 (e.g., at the distal end, or along the length, of the implant 140). In some embodiments, the implant 140 can be secured to the core member 132 via a surrounding sheath or other constraining mechanism that compresses the implant 140 into contact with the core member 140.

As best seen in FIG. 1B, the lead 124 and the core member 132 of the implant delivery assembly 130 can be coupled together via a surrounding sheath 118, which can take the form of a heat shrink tubing, a metal tube, a cable structure, a coil, or any other suitable structure that can surround and join together the lead 124 and the core member 132 along at least a portion of their respective lengths. In some implementations, the surrounding sheath 118 joins these elements together such that the lead 124 and core member 132 are not slidable relative to one another. In some embodiments, the sheath 118 can be configured to permit slidable movement of one or both of the lead 124 and the core member 132. The sheath 118 can be slidably disposed within the third catheter 114.

In some embodiments the implant 140 can be a tubular stent or other suitable implantable medical device. The implant 140 can have a low-profile, constrained or compressed configuration (not shown) for intravascular delivery to the treatment site within the third catheter 114, and an expanded configuration for securing and/or engaging a vessel wall and/or for restoring blood flow at the treatment site. In various embodiments, the implant 140 can take any number of forms, for example a tubular stent, a coiled wire, a weave, and/or a braid formed of a plurality of braided filaments. In the example illustrated in FIG. 1B, the implant 140 includes a generally tubular (e.g., cylindrical) body defining a working length WL along its distal portion, and a proximal portion of the implant 140 can taper proximally along a non-working length NWL to the connection point with the coupler 134. Examples of suitable implants 140 include any of those disclosed in U.S. Pat. No. 7,300,458, filed Nov. 5, 2007, U.S. Pat. No. 8,940,003, filed Nov. 22, 2010, U.S. Pat. No. 9,039,749, filed Oct. 1, 2010, and U.S. Pat. No. 8,066,757, filed Dec. 28, 2010, each of which is incorporated by reference herein in its entirety.

In some embodiments, the implant 140 is a mesh structure (e.g., a braid, a stent, etc.) formed of a superelastic material (e.g., Nitinol) or other resilient or self-expanding material configured to self-expand when released from the third catheter 114. In some variations, the implant 140 can be formed of or include any suitable material, including metals, Nitinol, stainless steel, platinum, gold, cobalt chromium, MP35N, suitable polymers, or any combinations thereof. Optionally, the implant 140 can be bioresorbable. In some embodiments, the implant 140 can be surface treated with a biomimetic material such as phosphorylcholine or other suitable material to avoid thrombogenic and/or embolic activity.

The implant 140 can be configured to provide sufficient radially outward force (also referred to as crush resistance) to treat venous stenosis, particularly in vessels in which the non-stenosed diameter is 3 mm or larger, 4 mm or larger, 5 mm or larger, or 6 mm or larger. In some implementations, the implant 140 can take the form of a hybrid implant with a combination of open and closed cells, which may offer comparatively higher radial force while maintaining sufficient flexibility to be positioned in tortuous vessels of the cerebral vasculature.

The implant 140 can be sized and configured for deployment in any intracranial venous structures, including but not limited to the transverse sinus, sigmoid sinus, superior/inferior sagittal sinus, internal cerebral vein, straight sinus or any other intracranial venous vessels or junctions between any of the aforementioned vessels. Additionally or alternatively, the implant 140 can be sized and configured for deployment in any extracranial venous structures deemed to be contributing to IIH, including but not limited to the internal or external jugular vein or maxillary vein.

Based on the dimensions of the vessels at the desired treatment site, the diameter of the implant 140 can be between about 3 mm and 12 mm, or between about 5 mm and 9 mm. some embodiments, the length of the implant 140 can be between 15 mm and 50 mm. In at least some implementations, the implant 140 can be slightly longer, when in the deployed state, than the axial length of the transition between the transverse sinus and the superior sagittal sinus.

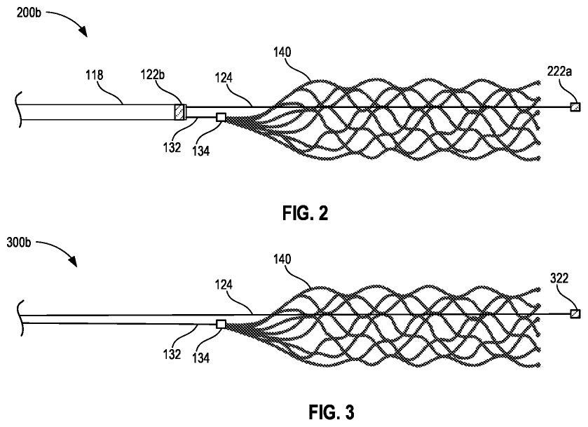

FIG. 2 is a side view of a distal portion 200b of an example treatment system 200 in accordance with the present technology. The treatment system 200 can include any of the features described previously with respect to the treatment system 100 above, for instance including a sheath 118 that surrounds both a lead 124 coupled to a sensor 222 and a core member 132 coupled to an implant 140. However, while the treatment system 100 shown in FIG. 1B depicts multiple sensors 122a and 122b coupled to one or more leads 124 that extend through the lumen of the sheath 118, the example shown in FIG. 2 includes a distal sensor 222a disposed at a distal end portion of the lead 124, and a proximal sensor 222b carried by the sheath 118 itself. The sensors 222 can be similar or identical to any of the sensors 122 described above.

In some variations, the proximal sensor 222b can be carried by a distal end portion of the third catheter 114 rather than the sheath 118, such that the distance between the proximal sensor 222b and the distal sensor 222a can be varied as the lead 124 slidably moves within the lumen of the third catheter 114. In such configurations, the lead 124 can be slidably advanced or retracted through the third catheter 114 relative to position the distal sensor 122a at various positions relative to the implant 140.

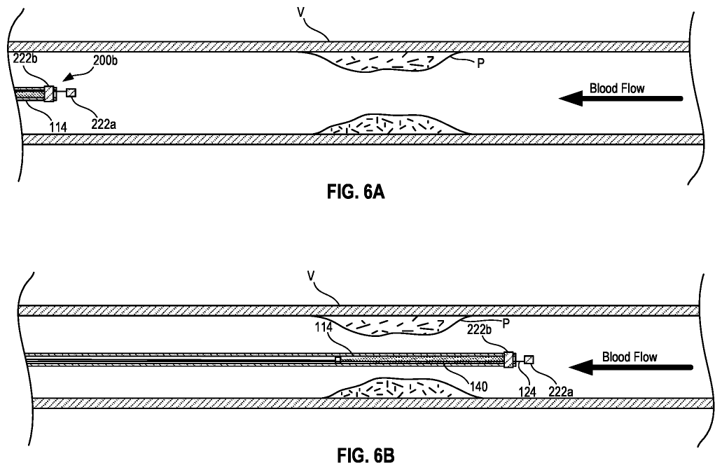

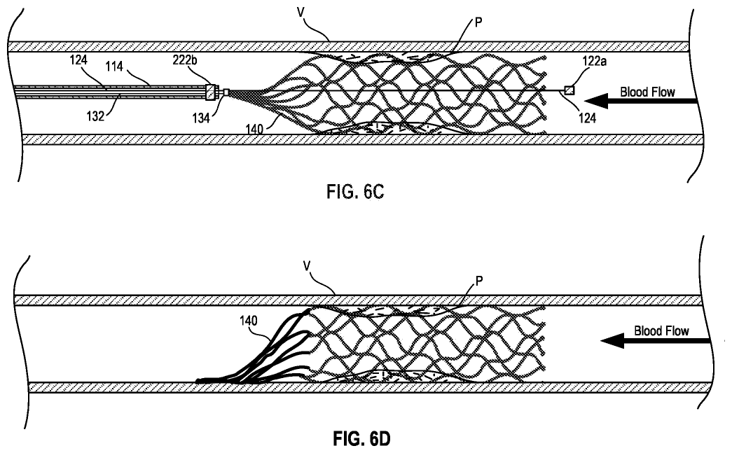

The proximal sensor 222b can be mounted to an outer or inner surface of the sheath 118 (or catheter 114). The sensors 222 can be similar to any of the sensors 122 described elsewhere herein. A separate lead or other electrical connection can be provided to couple to the proximal sensor 222b to the sensor electronics 126. Such electrical connection can be a separate wire or other conductive structure that extends proximally along the sheath 118, or alternatively may be integrated into the structure of the sheath 118 itself. Use of such a treatment system 200 is described in more detail below with respect to FIGS. 6A-6D.

FIG. 3 is a side view of a distal portion 300b of an example treatment system 300 in accordance with the present technology. The treatment system 300 can include any of the features described previously with respect to the treatment systems 100 and 200 above, for instance including a lead 124 coupled to a sensor 322 and a core member 132 coupled to an implant 140. However, while the treatment system 100 shown in FIG. 1B depicts multiple sensors 122a and 122b coupled to one or more leads 124 that extend through the lumen of the sheath 118, the example shown in FIG. 2 includes only a single sensor 322 disposed at a distal end portion of the lead 124. The sensor 322 can be similar or identical to any of the sensors 122, 222 described above. Additionally, while the treatment systems 100 and 200 described above include a sheath 118 that can join the core member 132 and lead 124 together in a non-slidable fashion, the sheath 118 can be omitted in the treatment system 300, such that the lead 124 is slidable relative to the core member 132. In this arrangement, the relative position of the sensor 322 and the implant 140 can be varied, such that a single sensor 322 can obtain measurements both proximal and distal to the implant 140. In such implementations, the lead 124 and the core member 132 can each extend through the lumen of the third catheter 114.

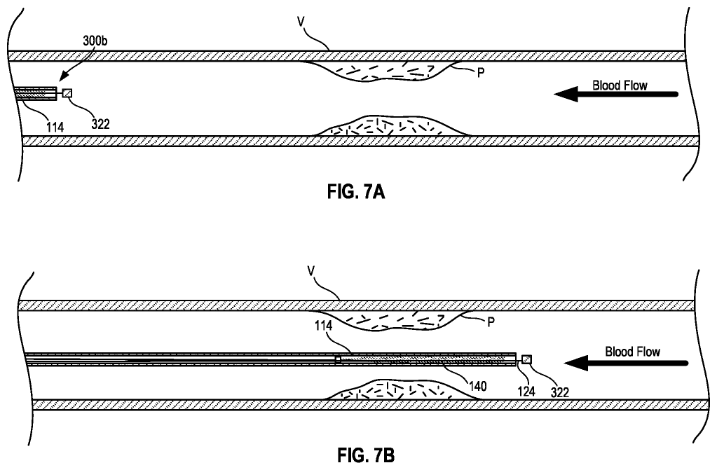

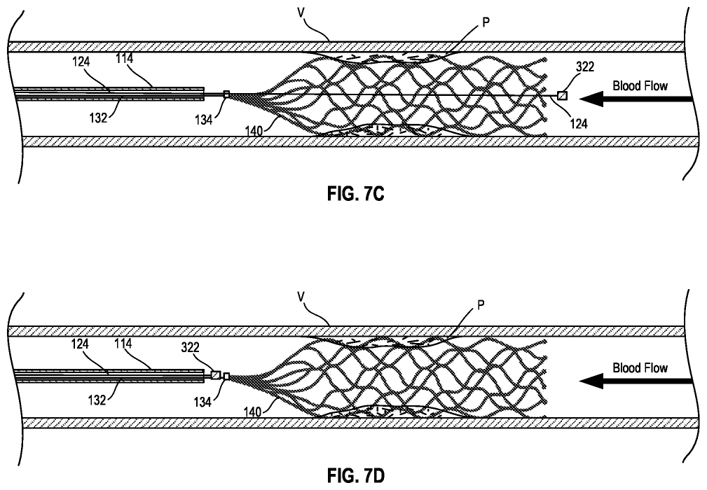

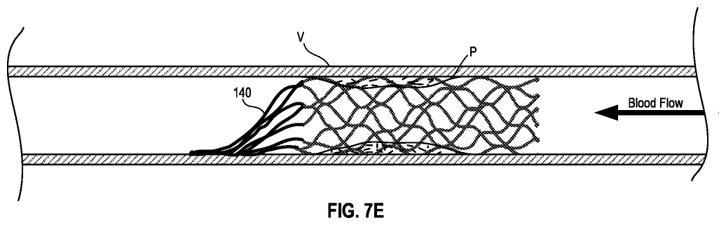

When using a system provided with a single sensor 322, following deployment of the implant it can be useful to position the sensor 322 at various locations relative to the implant 140 and/or the treatment site to obtain various in-vivo measurements. For instance, following deployment, the lead 124 can be slidably advanced or retracted through the catheter 114 to position the sensor 322 at various positions relative to the implant 140. Use of such a treatment system 300 is described in more detail below with respect to FIGS. 7A-7E.

While FIGS. 1B, 2, and 3 illustrate various configurations of sensors, other configurations are possible. For instance, one or more sensors can be positioned anywhere along the length of the implant 140, optionally being permanently or releasably mounted to the implant 140 itself. Such sensors may be positioned at locations near to radiopaque markers carried by the implant 140, such as markers positioned near a distal end of the implant 140 and/or the coupler 134 or other radiopaque structures disposed at a proximal end portion of the implant 140.

### II. Select Methods of Use

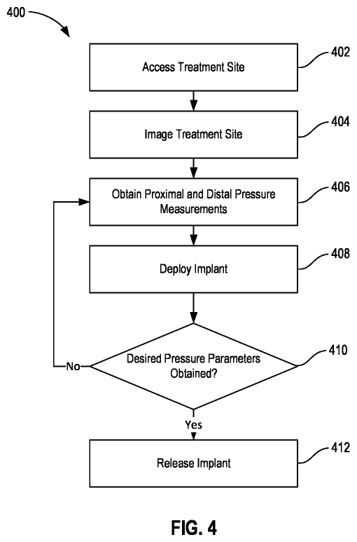

FIG. 4 is a flow chart illustrating a method 400 of treating intracranial stenosis in accordance with one or more embodiments of the present technology. The method 400 begins in block 402 with accessing the treatment site. For instance, in the case of intracranial venous stenosis, a treatment system (e.g., any one of the treatment systems 100, 200, or 300 described elsewhere herein) can be advanced intravascularly to the treatment site through the femoral vein, subclavian vein, internal jugular vein, or any suitable venous access site.

At block 404, the method 400 involves imaging the treatment site. This imaging can take the form of computed tomography (CT) venography, magnetic resonance venography, direct retrograde cerebral venography and manometry (DRCVM), or any other suitable modality configured to localize a stenosis. In various embodiments, this imaging can also include imaging the treatment system (e.g., using fluoroscopy) to confirm appropriate positioning of the delivery system with respect to the identified stenosis.

The method 400 continues in block 406 with obtaining proximal and distal pressure measurements. These pre-treatment measurements can be obtained using one or more sensors carried by a sensor assembly 120 of a treatment system 100, 200, or 300 as described elsewhere herein. This step can involve, for instance, advancing a treatment system to a treatment site and obtaining proximal and distal pressure measurements using the treatment site. In some embodiments, the proximal measurement is taken via a sensor positioned proximal to the stenosis and the distal measurement is taken via a sensor positioned distal to the stenosis. These pressure measurements may be used to calculate a parameter that characterizes blood pressure or flow at the treatment site. One example of such a parameter is a pressure ratio, in which a distal pressure measurement value is divided by a proximal pressure measurement value. Another example of such a parameter is a pressure differential, in which a distal pressure measurement value is subtracted from a proximal pressure measurement value (or vice versa). In the case of arterial vessels, the pressure ratio will generally be less than 1, as the blood pressure drops along the distal direction. In the case of venous vessels, the pressure ratio will generally be greater than 1, as the blood pressure increases along the distal direction. The magnitude of the ratio may indicate the presence of stenosis. For example, a stenosis can impede blood flow such that there is a significant change in pressure between the distal measurement (distal to the stenosis) and the proximal measurement (proximal to the stenosis). The larger the change across the stenosis, the greater the degree of blood flow impairment. Accordingly, determining a pressure ratio may both confirm the location of the stenosis and characterize its severity.

Following these measurements, in block 408 the implant is at least partially deployed. In some instances, the position of the implant can be confirmed under fluoroscopy or other imaging modality to ensure that the distal end portion of the implant is at or distal to the stenosis. The implant can be deployed by, for example, proximally retracting a surrounding catheter, distally advancing the implant relative to the surrounding catheter, or a combination thereof. In the case of a self-expanding tubular stent, moving the stent outside the catheter causes the stent to expand into apposition with the vessel wall at the stenosis. Depending on the configuration of the stent and the particular stenosis, the radially outward force of the stent expansion can increase patency of the blood vessel. In some embodiments, the implant can be deployed (e.g., moved into an expanded configuration) without being fully released, such that the implant may still be resheathed within the catheter if necessary.

In decision block 410, the method 400 involves determining whether the desired pressure parameters are obtained, such as by using a treatment system as described herein. For instance, additional proximal and/or distal pressure measurements can be obtained, and the values used to calculate appropriate parameters (e.g., pressure ratio post treatment). In some examples, a distal measurement can be obtained at a position distal to the implant, and a proximal measurement can be obtained at a position proximal to the distal position, such as along the working length of the implant, adjacent a proximal end portion of the implant, or proximal of the proximal end of the implant. Analyzing the pressure measurements at this stage can indicate whether blood flow has been improved and/or restored to acceptable levels. If it has not, then the clinician may decide to resheath and reposition the implant, or alternatively may resheath and remove the implant to be replaced with a different implant. In still other instances, the clinician may continue to release the current implant, and deliver a second implant in addition to the first to promote restoration of blood flow. In each of these cases, the method may return to block 406 to obtain proximal and distal pressure measurements before deploying (or re-deploying) an implant.

If, in block 410, the desired pressure parameters have been obtained, then the intervention has successfully restored or improved blood flow. In this scenario, the method 400 continues to block 412 with releasing the implant. This can involve, for example, severing a releasable connection to the implant via electrolytic detachment, mechanical detachment, or any other suitable detachment mechanism. While the implant remains in place within the body, the sensor assembly, catheter(s), and delivery system can be removed from the patient.

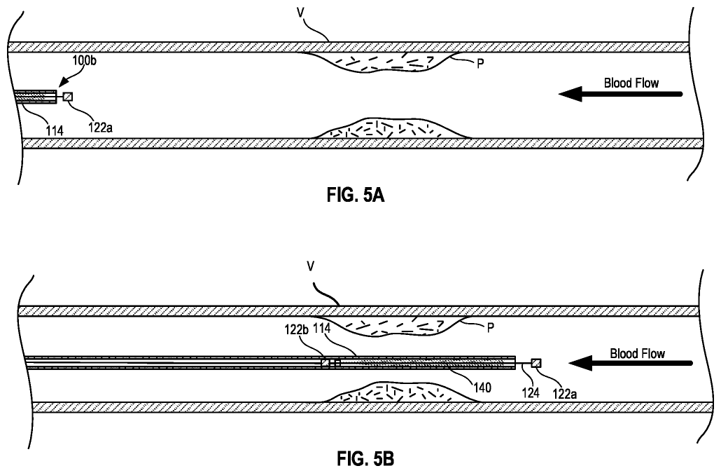

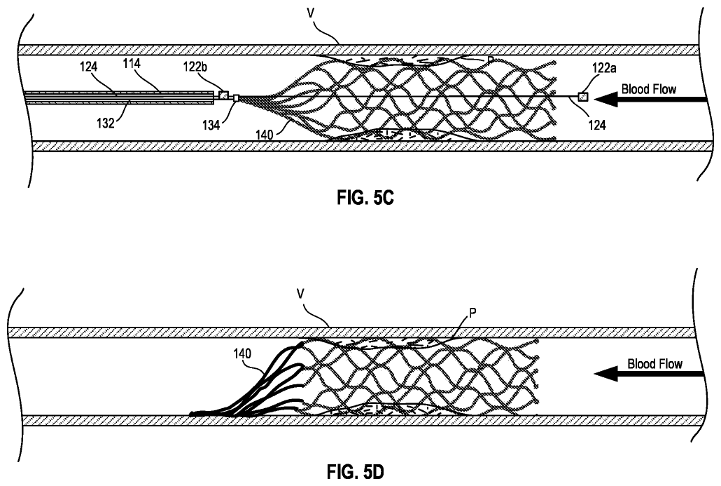

FIGS. 5A-5D illustrate an example method of treating intracranial stenosis in a blood vessel V using the treatment system 100 described above. As shown in FIG. 5A, the distal portion 100b of the treatment system 100 can be advanced toward an intravascular treatment site. In the illustrated example, the treatment site is a stenosed region of a vein, in which a buildup of plaque P within the lumen, has reduced the patency of the vein, thereby impeding blood flow therethrough. The cause of stenosis is not limited to the buildup of plaque P, and may be driven by other causes, such as enlargement of arachnoid granulations in the intracranial venous sinuses or extraluminal impingement of the intracranial venous sinuses, or any other known cause of stenosis. It is noted that plaque P is only one example of a cause of stenosis, and the systems and methods described herein can be used to resolve any underlying cause of stenosis. While in this configuration, the distal sensor 122a is exposed to blood flow in the vein, with the distal sensor 122a being disposed distal to the distal end of the third catheter 114. While the distal sensor 122a is positioned at a measurement location proximal to the stenosis, one or more measurements can be obtained via the distal sensor 122a. These measurements can indicate a proximal blood pressure (or other physiological characteristic at the proximal measurement location).

In the configuration shown in FIG. 5B, the distal portion 100b of the treatment system 100 has been further advanced distally such that the distal sensor 122a is positioned distal to the stenosis. The implant 140 remains in a low-profile, radially constrained configuration within the third catheter 114, and the proximal sensor 122b is also disposed within the lumen of the third catheter 114. As noted above, placement of the treatment system 100 to this location can be facilitated by imaging guidance (e.g., fluoroscopy), which may rely on visualizing radiopaque markers disposed along the implant 140, the sensors 122a and/or 122b, or other components of the treatment system 100. While the distal sensor 122a is positioned at a measurement location distal to the stenosis, one or more measurements can be obtained via the distal sensor 122a. These measurements can indicate a distal blood pressure (or other physiological characteristic at the proximal measurement location).

As noted previously, a pressure ratio may be calculated by dividing the distal pressure measurement by the proximal pressure measurement, and this pressure ratio can be indicative of the amount of blood flow through the stenosis. Accordingly, the measurement value obtained via the distal sensor 122a while in the position shown in FIG. 5B can be divided by the measurement value obtained via the distal sensor 122a while in the position shown in FIG. 5A to obtain a pressure ratio in the pre-treatment state.

Next, as illustrated in FIG. 5C, the third catheter 114 can be retracted and the implant 140 can self-expand into apposition with the wall of the vessel V at the stenosis. The radially outward force bears against at least a portion of the stenosis along the wall of the vessel V, thereby increasing the size of the lumen in that area and increasing patency of the blood vessel V. In this post-treatment state, additional measurements can be taken via the pressure sensors 122. Specifically, a post-treatment distal pressure measurement can be obtained via the distal sensor 122a and a post-treatment proximal pressure measurement can be obtained via the proximal sensor 122b. These values can be used to generate a post-treatment pressure ratio (or another suitable parameter).

As depicted in FIG. 5D, if the post-treatment pressure ratio indicates successful restoration of blood flow (e.g., the post treatment pressure ratio has dropped by more than a predetermined threshold amount compared to the pre-treatment pressure ratio), then the implant 140 can be released from the core member 132 (e.g., by severing the connection at the coupler 134), and the other components of the treatment system 100 can be retracted from the body, leaving the implant 140 in place at the stenosis. It is also noted that other embodiments of the delivery system may retain the ability to re-sheath the implant only as long as a proximal portion of the implant remains in the reduced diameter configuration while inside of catheter 114. Re-sheathing systems similar to those used on Medtronic's Pipeline Flex, Shield and Vantage flow diverter delivery systems are examples of such functionality, wherein the implant is re-sheathable as long as its proximal portion remains within the microcatheter used to deliver it.

FIGS. 6A-6D illustrate another example method of treating intracranial stenosis in a blood vessel V, in this instance using the treatment system 200 described above with respect to FIG. 2. As shown in FIG. 6A, the distal portion 200b of the treatment system 200 can be advanced toward an intravascular treatment site. In this configuration, both the distal sensor 222a (carried by the lead 124) and the proximal sensor 222b (carried by the third catheter 114 in this example) are positioned proximal to the stenosis. While in this configuration, one or more pre-treatment proximal measurements can be taken via one or both sensors 222a and 222b. These measurements can indicate a proximal blood pressure (or other physiological characteristic at the proximal measurement location).

In the configuration shown in FIG. 6B, the distal portion 200b of the treatment system 200 has been further advanced distally such that both the distal sensor 222a and the proximal sensor 222b are positioned distal to the stenosis. The implant 140 remains in a low-profile, radially constrained configuration within the third catheter 114. In this arrangement, one or more pre-treatment distal measurements can be taken via one or both of the sensors 222a and 222b. These measurements can indicate a distal blood pressure (or other physiological characteristic at the proximal measurement location). As noted previously, a pre-treatment pressure ratio may be calculated by dividing the distal pressure measurement by the proximal pressure measurement, and this pressure ratio can be indicative of the amount of blood flow through the stenosis.

Next, as illustrated in FIG. 6C, the third catheter 114 can be retracted and the implant 140 can self-expand into apposition with the wall of the vessel V at the stenosis. In this post-treatment state, additional measurements can be taken via the pressure sensors 222. Specifically, a post-treatment distal pressure measurement can be obtained via the distal sensor 222a (carried by the lead 124) and a post-treatment proximal pressure measurement can be obtained via the proximal sensor 222b (carried by the third catheter 114). These values can be used to generate a post-treatment pressure ratio (or another suitable parameter).

As depicted in FIG. 6D, if the post-treatment pressure ratio indicates successful restoration of blood flow (e.g., the post treatment pressure ratio has decreased by more than a predetermined threshold amount compared to the pre-treatment pressure ratio), then the implant 140 can be released from the core member 132 (as discussed above), and the other components of the treatment system 100 can be retracted from the body, leaving the implant 140 in place at the stenosis.

FIGS. 7A-7E illustrate another example method of treating intracranial stenosis, here using the treatment system 300 described previously with respect to FIG. 3. As discussed above, the treatment system 300 includes only a single sensor 322 mounted to a lead 124, with the lead being slidable relative to the core member 132 and the expanded implant 140. As such, the single sensor 322 can be moved to different locations relative to the implant 140 to facilitate obtaining pre-treatment and post-treatment measurements in proximal and distal locations.

As shown in FIG. 7A, the distal portion 300b of the treatment system 300 can be advanced toward an intravascular treatment site, where the sensor 322 can obtain a pre-treatment proximal measurement of blood pressure and/or blood flow. Next, as shown in FIG. 7B, the distal portion 300b of the treatment system 300 can be further advanced distally until the sensor 322 is positioned distal to the stenosis, at which point a distal pre-treatment measurement can be obtained. In this stage, the implant 140 remains in a low-profile, radially constrained configuration within the third catheter 114. As noted above, a pressure ratio may be calculated by dividing the distal pressure measurement by the proximal pressure measurement, and this pre-treatment pressure ratio can be indicative of the amount of blood flow through the stenosis.

Next, as illustrated in FIG. 7C, the third catheter 114 can be retracted and the implant 140 can self-expand into apposition with the wall of the vessel V at the stenosis. In this post-treatment state, additional measurements can be taken via the pressure sensor 322, which is positioned distal to the stenosis and distal to the implant 140. This provides a post-treatment distal pressure measurement.

The sensor 322 can then be proximally retracted through the lumen of the implant 140 to a position proximal to the implant 140, as shown in FIG. 7D. In this state, the sensor 322 can obtain a post-treatment proximal pressure measurement. Together, the post-measurement values obtained in the arrangements shown in FIGS. 7C and 7D can be used to generate a post-treatment pressure ratio (or another suitable parameter).

Finally, as depicted in FIG. 7E, if the post-treatment pressure ratio indicates successful restoration of blood flow (e.g., the post treatment pressure ratio has decreased by more than a predetermined threshold amount compared to the pre-treatment pressure ratio), then the implant 140 can be released from the core member 132 (as described above), and the other components of the treatment system 100 can be retracted from the body, leaving the implant 140 in place at the treatment site.

As noted previously, the treatment systems described herein can advantageously enable a series of pre-and post-treatment measurements of blood flow and/or blood pressure at the site of venous stenosis or other treatment sites. These measurements may be aggregated across multiple patients to create a combined dataset (appropriately anonymized) that relates these values to a number of other parameters, such as pre-, intra-, and/or post-procedural patient conditions, geometry of patient vessels, patient demographic information, medical history, symptom information, etc. Such combined datasets may be useful in developing predictive algorithms (e.g., using artificial intelligence such as deep learning, machine learning, or other suitable techniques). In some implementations, the aggregated data enable clinicians to identify and/or predict comorbidities or risk factors related to IIH. Moreover, such analysis can provide intra-and post-procedure confirmation of treatment efficacy, intra-procedural recommendations regarding repositioning of the implant, intra-procedural recommendations regarding replacement of a given implant with a different size, and other intra-procedural feature treatment requirements based on some or all or the aggregated data. In various embodiments, such feedback, guidance, or warnings may be presented to users or clinicians via a mobile application, web site, or other suitable avenue.

### III. Conclusion

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may have been disclosed in the context of those embodiments, other embodiments can also exhibit such advantages, and not all embodiments need necessarily exhibit such advantages or other advantages disclosed herein to fall within the scope of the present technology. Accordingly, this disclosure and associated technology can encompass other embodiments not expressly shown and/or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

1. A method for treatment of intracranial stenosis comprising:
disposing a catheter within a blood vessel such that a distal end of the catheter is disposed proximal to a treatment site, the catheter defining a lumen containing a stent in a radially compressed configuration;
disposing a pressure sensor at or distal to a distal end of the catheter at a proximal measurement site that is proximal to the treatment site;
obtaining a pre-treatment proximal blood pressure parameter via the pressure sensor at the proximal measurement site;
disposing a pressure sensor at a distal measurement site that is distal to the treatment site;
obtaining a pre-treatment distal blood pressure parameter via the pressure sensor at the distal measurement site;
deploying the stent by:
while the distal end of the catheter is disposed at or distal to the treatment site, proximally retracting the catheter relative to the stent, thereby releasing the stent from the catheter; and
allowing the stent to self-expand into apposition with the blood vessel at the treatment site;
obtaining a post-treatment distal blood pressure parameter via a pressure sensor at the distal measurement site; and
obtaining a post-treatment proximal blood pressure parameter via a pressure sensor at the proximal measurement site.

2. The method of claim 1, wherein a single pressure sensor is used to obtain the pre-treatment proximal blood pressure parameter, the post-treatment proximal blood pressure parameter, the pre-treatment distal blood pressure parameter, and the post-treatment distal blood pressure parameter, wherein the pressure sensor is coupled to an elongate member slidably extending through the catheter lumen.

3. The method of claim 2, wherein obtaining the pre-treatment distal blood pressure parameter comprises distally advancing the elongate member such that the pressure sensor is moved from the proximal measurement site to the distal measurement site.

4. The method of claim 2, wherein obtaining the post-treatment proximal blood pressure parameter comprises proximally retracting the elongate member such that the pressure sensor is moved from the distal measurement site to the proximal measurement site.

5. The method of claim 1, wherein:
a proximal pressure sensor is used to obtain the pre-treatment proximal blood pressure parameter and to obtain the post-treatment proximal blood pressure parameter,
a separate distal pressure sensor is used to obtain the pre-treatment distal blood pressure parameter and the post-treatment distal blood pressure parameter, and
the distal pressure sensor is coupled to an elongate member slidably extending through the catheter lumen.

6. The method of claim 5, wherein the proximal pressure sensor is carried by the catheter.

7. The method of claim 5, wherein the proximal pressure sensor is carried by the elongate member at a position proximal to the distal pressure sensor.

8. The method of claim 1, further comprising:
comparing the pre-treatment and post-treatment blood pressure parameters; and
based on the comparison, re-positioning the stent within the blood vessel.

9. A system for treatment of intracranial stenosis comprising:
a catheter defining a lumen and having a distal end portion configured to be disposed adjacent to a treatment site within an intracranial blood vessel;
an elongate member extending through the catheter lumen;
a distal pressure sensor coupled to a distal end portion of the elongate member, the distal pressure sensor configured to obtain blood pressure parameters to characterize blood flow at the treatment site before and after treatment; and
a stent disposed within the catheter lumen in a radially constrained configuration, the stent surrounding the elongate member such that the distal end portion of the elongate member is disposed distal to a distal end of the stent, wherein the stent is configured to radially expand into apposition with the blood vessel to increase blood flow at the treatment site when released from the catheter.

10. The system of claim 9, further comprising a proximal pressure sensor configured to obtain blood pressure parameters in conjunction with the distal pressure sensor to characterize blood flow at the treatment site before and after treatment.

11. The system of claim 10, wherein the proximal pressure sensor is carried by the elongate member at a position proximal to the distal pressure sensor.

12. The system of claim 10, wherein the proximal pressure sensor is carried by the catheter.

13. The system of claim 9, further comprising:
a second elongate member extending through the catheter lumen; and
a proximal pressure sensor coupled to a distal end portion of the second elongate member, the proximal pressure sensor configured to obtain blood pressure parameters in conjunction with the distal pressure sensor to characterize blood flow at the treatment site before and after treatment.

14. The system of claim 9, further comprising:
a pusher member releasably coupled to the stent and extending through the catheter lumen; and
a second sheath disposed within the catheter lumen, the second sheath surrounding the pusher member and at least a proximal portion of the elongate member.

15. A method of treating intracranial stenosis with the system of claim 9, the method comprising:
distally advancing the catheter carrying the elongate member and the stent through a patient's vasculature to a position proximal to treatment site within an intracranial blood vessel, such that the distal pressure sensor extends beyond a distal end of the catheter and is disposed at a proximal measurement site that is proximal to the treatment site;
obtaining a pre-treatment proximal blood pressure parameter via the distal pressure sensor at the proximal measurement site;
distally advancing the catheter and the elongate member to position the distal pressure sensor at a distal measurement site distal that is distal to the treatment site;
obtaining a pre-treatment distal blood pressure parameter via the distal pressure sensor at the distal measurement site;
deploying the stent by proximally retracting the catheter relative to the stent, thereby releasing the stent from the catheter such that the stent self-expands into apposition with the blood vessel at the treatment site;
after deploying the stent, obtaining a post-treatment distal blood pressure parameter via the distal pressure sensor at the distal measurement site;
proximally retracting the elongate member to position the distal pressure sensor at the proximal measurement site; and
obtaining a post-treatment proximal blood pressure parameter via the distal pressure sensor at the proximal measurement site.

16. The method of claim 15, wherein the stent is releasably coupled to a pusher member, the method further comprising, after obtaining the post-treatment proximal blood pressure parameter and obtaining the post-treatment distal blood pressure parameter, releasing the stent from the pusher member.

17. A method of treating intracranial stenosis with the system of claim 9, the method comprising:
distally advancing the catheter carrying the elongate member and the stent through a patient's vasculature to a treatment site within an intracranial blood vessel, such that the distal pressure sensor extends beyond a distal end of the catheter and is disposed at a proximal measurement site that is proximal to the treatment site;
obtaining a pre-treatment proximal blood pressure parameter via the distal pressure sensor at the proximal measurement site;
distally advancing the catheter and the elongate member to position the distal pressure sensor at a distal measurement site distal that is distal to the treatment site;
obtaining a pre-treatment distal blood pressure parameter via the distal pressure sensor at the distal measurement site;
deploying the stent by proximally retracting the catheter relative to the stent, thereby releasing the stent from the catheter such that the stent self-expands into apposition with the blood vessel at the treatment site;
after deploying the stent, obtaining a post-treatment distal blood pressure parameter via the distal pressure sensor at the distal measurement site;
disposing a proximal pressure sensor at the proximal measurement site; and
after deploying the stent, obtaining a post-treatment proximal blood pressure parameter via the proximal pressure sensor at the proximal measurement site.

18. The method of claim 17, wherein the proximal pressure sensor is carried by the elongate member at a position proximal to the distal pressure sensor.

19. The method of claim 17, wherein the proximal pressure sensor is carried by a second elongate member slidably disposed within the catheter.

20. The method of claim 17, wherein the proximal pressure sensor is carried by the catheter.

21. The method of claim 17, wherein the stent is releasably coupled to a pusher member, the method further comprising, after obtaining the post-treatment proximal blood pressure parameter and obtaining the post-treatment distal blood pressure parameter, releasing the stent from the pusher member.
